(19) United States
(12) Patent Application Publication
(10) Pub. No.: US 2014/0084949 A1
(43) Pub. Date: Mar. 27, 2014

(54) SURFACE IMPEDANCE SYSTEMS AND METHODS

(71) Applicant: Access Business Group International LLC, Ada, MI (US)

(72) Inventors: Matthew T. Smith, Wyoming, MI (US); David A. Meekhof, Grand Rapids, MI (US); Richard B. Bylsma, Ada, MI (US); David J. Anderson, Ada, MI (US)

(73) Assignee: Access Business Group International LLC, Ada, MI (US)

(21) Appl. No.: 14/022,483

(22) Filed: Sep. 10, 2013

Related U.S. Application Data
(60) Provisional application No. 61/704,713, filed on Sep. 24, 2012.
(60) Provisional application No. 61/789,495, filed on Mar. 15, 2013.

(51) Int. Cl. A61B 8/00; G01B 7/28; A61N 7/00

(52) U.S. Cl. CPC A61B 8/4272; A61N 7/00; G01B 7/28
USPC 324/693; 601/2

(57) ABSTRACT

A surface impedance sensor and method are provided. The surface impedance sensor generally includes first and second electrodes, a driver circuit to drive the electrodes at a plurality of driving frequencies, and a detection circuit to measure the impedance across the first and second electrodes for comparison against a plurality of reference profiles. The method generally includes measuring the localized surface impedance for each of a plurality of driving frequencies to generate a measured profile, and correlating the measured profile with a reference profile. The system and method can verify contact with a particular surface and can be used with a variety of host devices, including for example ultrasound delivery devices.

### FIELD OF THE INVENTION

The present invention relates to surface impedance systems, and more particularly, to surface impedance systems for ultrasound devices and other applications.

### BACKGROUND OF THE INVENTION

Ultrasound devices are widely used as a diagnostic aid and, more recently, as therapeutic tools, and in particular, a treatment aid for the rejuvenation of the skin. Known devices typically include an ultrasound transducer within a handpiece for propagating targeted ultrasonic energy toward the body. To enhance the acoustic coupling between the ultrasound transducer and the body, a transduction gel having desired acoustic properties is typically applied to the exposed skin before operation of the transducer.

Typical transduction gels are sufficiently viscous to eliminate the presence of air pockets between the transducer and the skin. In addition, typical transduction gels are acoustically similar to that of skin tissue to minimize the reflection of ultrasonic energy at the gel-skin interface. While there exists a variety of known methods for applying a transduction gel to the skin, perhaps the most common method involves the manual application and distribution of a transduction gel to an ultrasound focus area.

While simplistic, the above known method is prone to variations based on the experience and skill of the person applying the transduction gel. Particularly with untrained persons, the application of transduction gel can be insufficient, leaving air pockets between the transducer and the skin, or wasteful, consuming excessive quantities of transduction gel. Accordingly, there remains a need for an improved system and method for the application of transduction gel to the skin, and in particular, an improved system and method for detecting sufficient quantities of transduction gel on the skin prior to and during application of ultrasonic energy to the body.

### SUMMARY OF THE INVENTION

A surface impedance sensor and method are provided. In a first aspect of the invention, the surface impedance sensor includes first and second electrodes, a driver circuit to drive the electrodes at a plurality of driving frequencies, and a detection circuit to measure the impedance across the first and second electrodes for comparison against a plurality of reference profiles. The surface impedance sensor can additionally include a controller to correlate the measured impedance with one of the plurality of reference profiles stored in memory. The controller can optionally provide an output indicative of the presence or absence of a particular surface in contact with the electrodes.

In one embodiment, the detection circuit is adapted to measure the complex impedance across the first and second electrodes for each of the plurality of driving frequencies. The reference profiles are stored in memory and correspond to either a transduction gel or bare skin. The reference profiles can include an impedance curve that begins at a first asymptotic value at relatively low driving frequencies and transitions to a second, lesser asymptotic value at relatively high driving frequencies.

In another embodiment, the surface impedance sensor is housed within an ultrasound delivery device. In this embodiment, the first and second electrodes are translucent to ultrasonic energy, and the controller output is used to control application of ultrasonic energy to the skin. Optionally, the ultrasound delivery device includes a gel dispenser that regulates the application of gel to the skin based on the controller output.

In another aspect of the invention, a method is provided for distinguishing among skin, a gel or a foreign object. The method generally includes applying first and second electrodes to a surface portion, driving the first and second electrodes at a plurality of driving frequencies, measuring the localized surface impedance for each of the plurality of driving frequencies to generate a measured profile, and correlating the measured profile with a reference profile to identify the surface portion.

In one embodiment, the method includes measuring the complex impedance across the first and second electrodes for each of the plurality of driving frequencies. The measured profile can include a frequency response curve for the local surface impedance that begins at an upper impedance value and declines toward a lower impedance value. The upper and lower values differ among each of the possible surfaces to permit the real time discrimination among possible surfaces.

In another embodiment, the method includes providing an output to a handheld ultrasound delivery device. The ultrasound delivery device can include a transducer adapted to provide a focused line of ultrasonic energy if a sufficient quantity of transduction gel is in contact with the electrodes. In addition, the ultrasound delivery device can include an on-board transduction gel dispenser to discharge regulated transduction gel quantities at the skin surface.

In still another aspect of the invention, a skin contact sensor is provided. The skin contact sensor includes a driver circuit adapted to generate a pulsed voltage across first and second electrodes, a measurement circuit adapted to measure a characteristic of the pulsed voltage across the first and second electrodes, and a controller coupled to the measurement circuit and adapted to determine the identity of the surface in contact with the electrodes based on the measured characteristic.

In one embodiment, the driver circuit applies a pulsed signal to the first electrode. The pulsed signal includes a repeating square wave having a frequency of between approximately 0.1 kHz and 10 kHz, a pulse width of between approximately 50 microseconds and 5 milliseconds, and a peak voltage between approximately 0.5V and about 10V. The measurement circuit then samples a pulsed voltage at the second electrode, which is somewhat distorted when compared to the original pulsed signal.

In another embodiment, the measurement circuit is adapted to determine first and second characteristics of the pulsed voltage. The first characteristic includes the difference between the first and last non-zero portions of the pulsed voltage. The second characteristic includes the sum of certain non-zero portions of the pulsed voltage. The controller is adapted to rapidly verify contact with a particular surface based on a real-time comparison of these characteristics with predetermined baselines.

Embodiments of the invention can therefore provide an improved sensor and method to verify contact with a particular surface based on: (a) a real-time comparison between measured impedance values and reference impedance values across a range of driving frequencies; and/or (b) a real-time comparison between measured pulse characteristics with baseline values for different surfaces. The sensor and method can be used in combination with a variety of host devices, including for example ultrasound delivery devices, vehicle door handles, and trip sensors for heavy machinery. When used in combination with ultrasound delivery devices, the sensor and method can reduce or eliminate variations in gel levels otherwise attributable to the user, and can instead provide the consistent application of a transduction gel before and during operation of the ultrasonic delivery device.

These and other advantages and features of the invention will be more fully understood and appreciated by reference to the description of the current embodiments and the drawings.

### BRIEF DESCRIPTION OF THE DRAWINGS

FIG. 1 is a schematic representation of a first surface impedance sensor.

FIG. 2 is a circuit diagram of a complex impedance detection circuit.

FIG. 3 is a schematic representation of a second surface impedance sensor.

FIG. 4 is a circuit diagram of a resistive impedance detection circuit.

FIG. 5 is a flow chart illustrating a method of the present invention.

FIG. 6 is a graph illustrating impedance profiles for multiple aqueous solutions.

FIG. 7 is a graph illustrating impedance profiles for skin with and without aqueous solutions.

FIG. 8 is a graph illustrating an impedance profile for an electrode gel.

FIG. 9 is a graph illustrating an impedance profile for dry skin.

FIG. 10 is a graph illustrating an impedance profile for a milled wood surface.

FIG. 11 is an illustration of an ultrasound delivery device.

FIG. 12 is an illustration of a first acoustic nose assembly tip.

FIG. 13 is an illustration of a second acoustic nose assembly tip.

FIG. 14 is a schematic representation of a skin contact sensor.

FIG. 15 is a graph illustrating the measured pulsed voltage across first and second electrodes of the skin contact sensor of FIG. 14 for a single surface portion.

FIG. 16 is a graph illustrating the measured pulsed voltage across first and second electrodes of the skin contact sensor of FIG. 14 for multiple surface portions.

FIG. 17 is a flow chart illustrating a method of operating the skin contact sensor of FIG. 14.

FIG. 18 is a classification graph including the slope and the area of measured pulse voltages for multiple surface portions.

FIG. 19 is a classification graph for a skin contact sensor having corroded electrodes.

FIG. 20 is a classification graph for a skin contact sensor having 1.0 mm electrodes.

FIG. 21 is a classification graph for a skin contact sensor having 0.5 mm electrodes.

### DESCRIPTION OF THE CURRENT EMBODIMENTS

The current embodiments relate to a system and a method for verifying contact with a surface based on (a) a comparison between a measured impedance profile and a reference impedance profile, discussed in Part I below, or (b) a classification of measured pulse characteristics, discussed in Part II below. The system and method of the present invention can be implemented across a range of applications where it is desirable to rapidly verify contact with a particular surface or object, including for example applications involving the detection of transduction gels and/or skin tissue.

### I. Impedance Profile Comparison

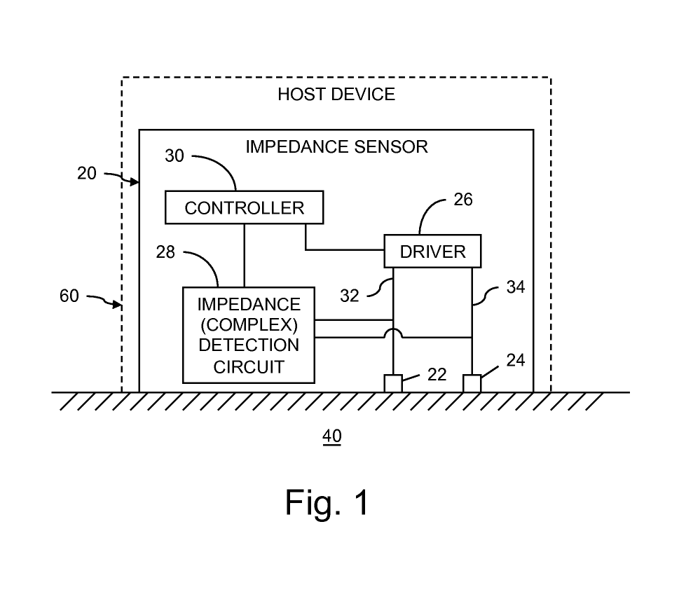

Referring now to FIG. 1, a first surface impedance sensor in accordance with an embodiment of the invention is illustrated and generally designated 20. The surface impedance sensor 20 includes first and second electrodes 22, 24, a driver circuit 26, an impedance detection circuit 28, and a controller 30. The first and second electrodes 22, 24 are initially electrically isolated from each other, optionally being separated by a fixed distance. The driver circuit 26 is electrically coupled to one or both of the first and second electrodes 22, 24 to drive the first and second electrodes 22, 24 with a time-varying current at a plurality of frequencies. The time-varying current is optionally an alternating current, for example a sine wave, a square wave, or a sawtooth wave. The driving circuit 26 of the present embodiment is adapted to drive the electrodes with a sinusoidal current between about 10 Hz and about 1 MHz. The driving circuit 26 can alternatively be adapted to drive the electrodes across a frequency range that includes substantially less than 10 Hz and/or substantially greater than 1 MHz, including for example 1 Hz and 10 MHz, and further by example 0.1 Hz and 100 MHz.

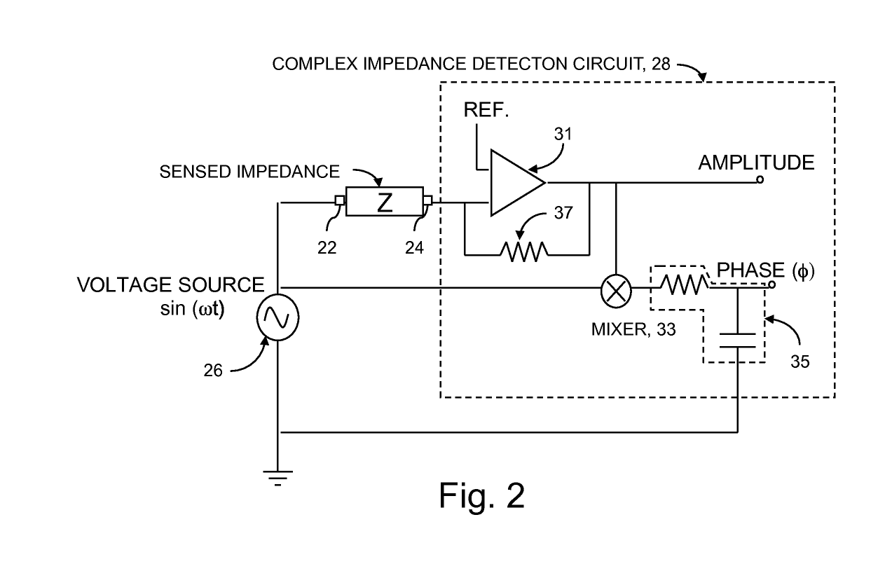

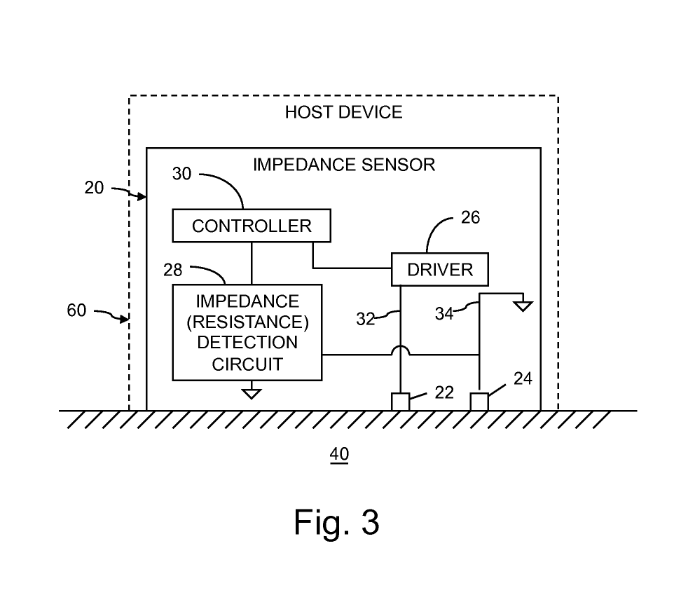

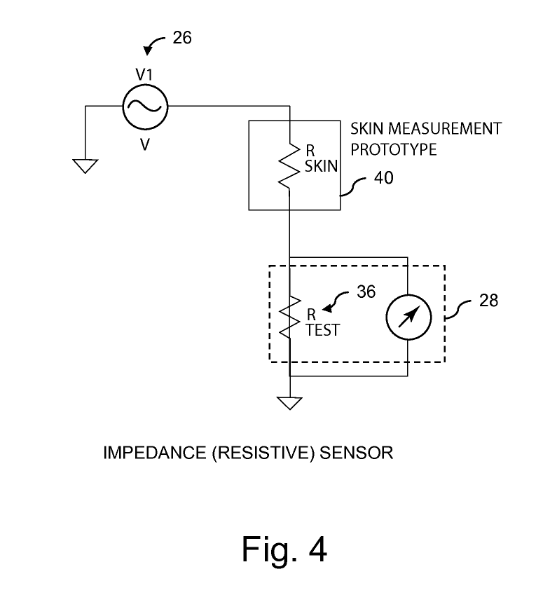

As noted above, the impedance sensor 20 includes an impedance detection circuit 28 to measure a local surface impedance between the first and second electrodes 22, 24. Because the local surface impedance is in many instances frequency dependent, the impedance detection circuit 28 can measure the local surface impedance for each driving frequency. The impedance detection circuit 28 can include analog or digital processing to determine one or both of a reactance and a resistance. For example, a complex impedance detection circuit 28 can be coupled to both electrode leads 32, 34 to directly or indirectly measure (a) the amplitude of the voltage (or current) across the electrodes and (b) the phase between the current and voltage across the electrodes 22, 24. As shown in FIG. 2, an exemplary complex impedance detection circuit 28 can include a differential amplifier 31, a mixer 33, and a low pass filter 35. The differential amplifier 31 can include an inverted input coupled in series with the electrodes 22, 24, a non-inverted input coupled to a reference voltage (Ref.), and a resister 37 setting the amplifier gain. In this configuration, the amplifier output is proportional to the difference between the voltage across the electrodes 22, 24 and the reference voltage (Ref.). In addition, the output of the amplifier is mixed with the output of the source voltage to indirectly determine the phase across the first and second electrodes 22, 24. The low pass filter 37 then shunts high frequency signals to ground, providing a DC output corresponding to the phase difference. As a result, the exemplary complex impedance detection circuit 28 provide an “amplitude” analog output and a “phase” analog output to the controller 30. The controller 30 can then include an analog to digital converter and digital signal processing to determine the complex impedance for a given driving frequency. Also by example, the impedance detection circuit 28 can be coupled to a single electrode lead 32 to measure only the amplitude of the voltage (or current) across a resister 36 in series with the first and second electrodes 22, 24 as shown in FIGS. 3-4. In these embodiments, the impedance detection circuit 28 provides an output based on the resistive impedance of the local surface impedance for each driving frequency. The controller 30, in turn, accepts the output and generates a measured impedance profile over successive impedance measurements. The controller 30 can optionally include an analog to digital converter and digital signal processing to correlate the measured impedance profile with one or more reference impedance profiles. For example, the controller 30 can include multiple reference impedance profiles stored in memory and corresponding to multiple gel formulations and multiple skin types. The controller 30 can provide an output to a host device 60 to indicate the absence or presence of a particular gel formulation in contact with the electrodes 22, 24. The host device 60, in turn, can activate a transducer if transduction gel is detected or a gel dispenser if only skin is detected.

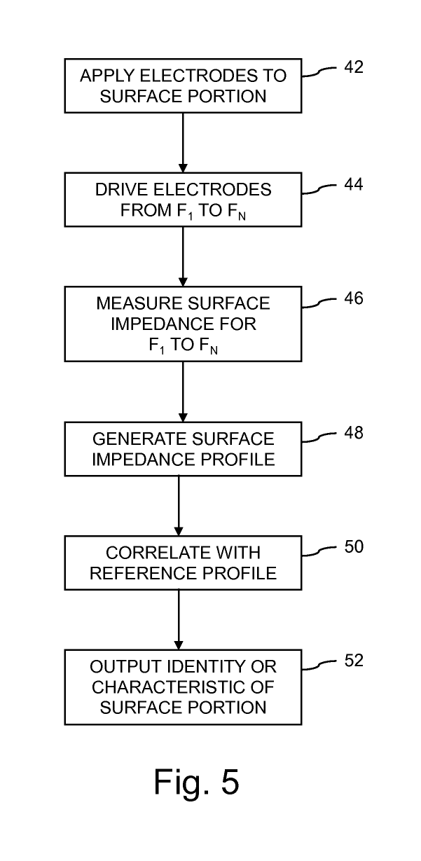

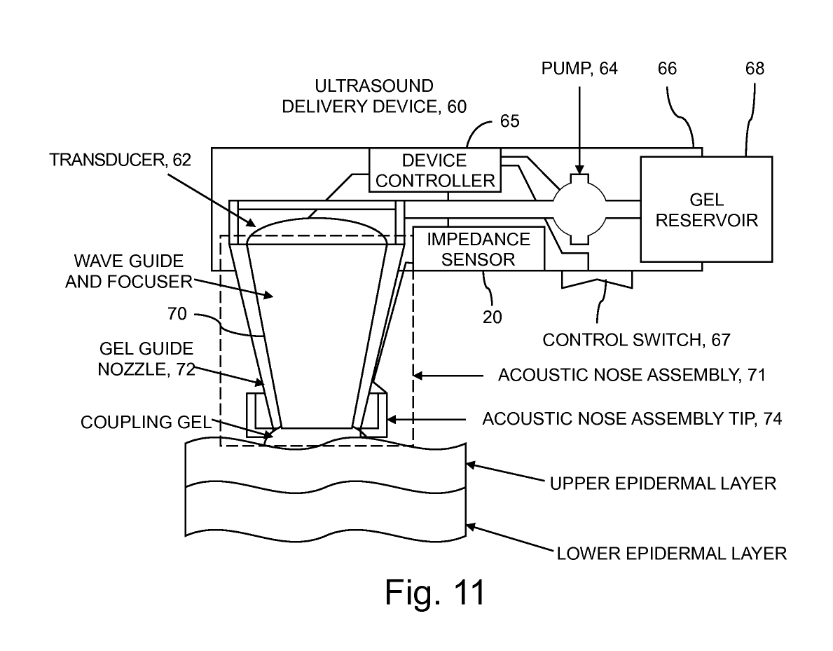

A flow chart illustrating a method for operating the impedance sensor of FIG. 1 is shown in FIG. 5. The method includes applying the electrodes 22, 24 to a surface portion 40 at step 42. The surface portion 40 completes the electrical circuit between the electrodes 22, 24, which are otherwise electrically isolated from each other, optionally being spaced apart by a fixed distance. This surface portion 40 can be any material having an impedance, including for example materials that are dimensionally stable at room temperature and pressure and materials that are non-dimensionally stable at room temperature and pressure. At step 44, the driver circuit 26 passes a time-varying current from the first electrode 22 to the second electrode 24 through the surface portion 40, and at plurality of driving frequencies, denoted F₁ to F_{N}. Optionally, the driving frequencies include about 10 Hz to about 1 MHz at regular or irregular intervals. At step 46, the impedance measuring circuit 28 determines the local impedance for each driving frequency. The local impedance can include the complex impedance, e.g., the reactance and the resistance, the reactance only, or the resistance only. Though shown as separate steps, steps 44 and 46 are interleaved operations. In other words, the detection circuit 28 determines an impedance value at F₁ before the driver 26 adjusts the driving frequency to F₂, optionally under the control of the controller 30. The measured impedance values accumulated by the controller 30 are used to generate a measured surface impedance profile at step 48. As explained in more detail below, the surface impedance profile can include a curve that transitions from a high impedance value at low frequencies to a low impedance value at high frequencies. At step 50, the measured surface impedance profile is correlated with a reference surface impedance profile, optionally by the controller 30. The reference surface impedance profile can correspond to the perceived identity of the surface portion, including for example a particular gel formulation or skin tissue. At step 52, an identifier associated with the relevant reference surface impedance profile is provided to a host device 60. This identifier can be used, for example, to control an ultrasound delivery device as discussed more fully in connection with FIGS. 11-13 below.

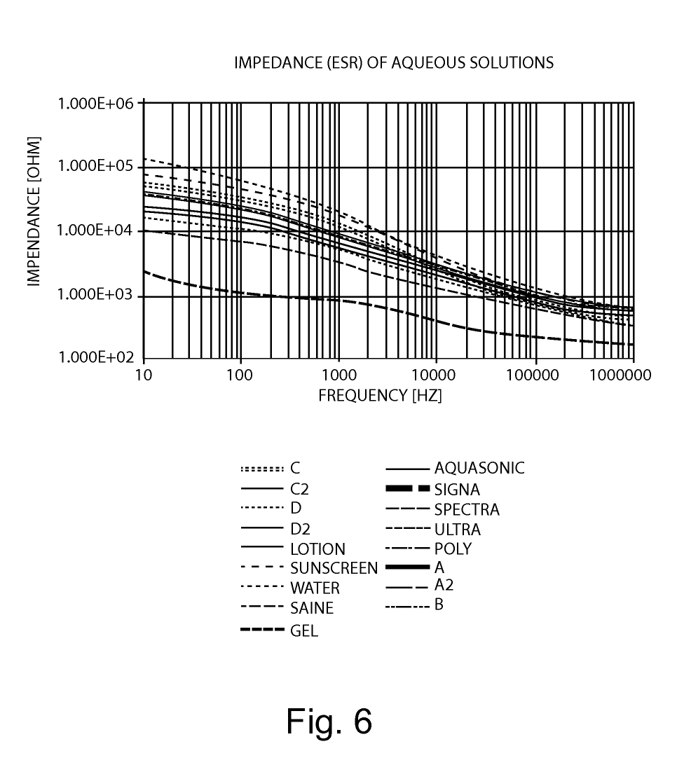

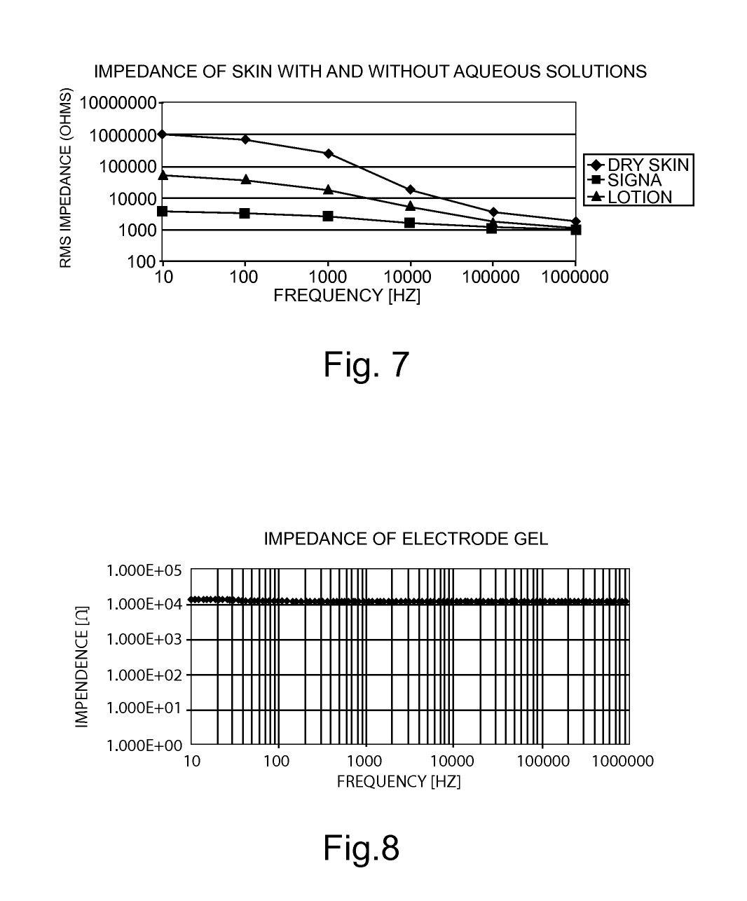

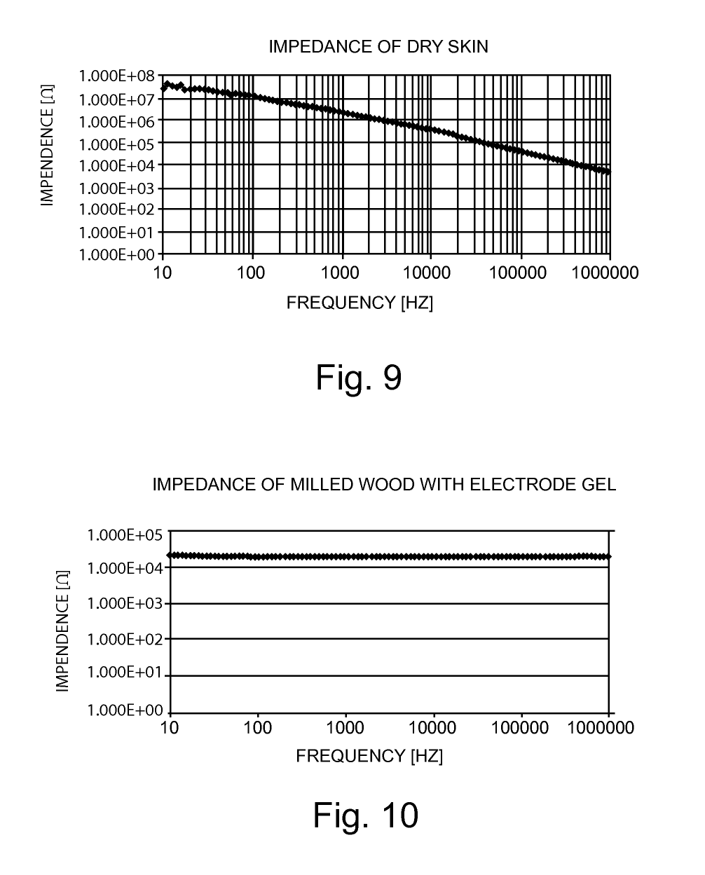

Referring now to FIG. 6, exemplary impedance profiles are depicted on a log-log plot for a variety of aqueous solutions, including electrode gel formulations, a lotion, a sunscreen, water and a saline. The electrodes were driven at a range of frequencies from about 10 Hz to about 1 MHz, inclusive. The impedance profiles were obtained using an LCR meter coupled to a 1 cm×1 mm electrode pair spaced 2 cm apart. Each solution exhibited a discrete low frequency impedance that trended asymptotically to a (nearly) common high frequency impedance. The low frequency impedance values varied from about 2E3 Ohms (electrode gel) to about 1.1E5 Ohms (water) while the high frequency impedance values varied from about 2E2 Ohms (electrode gel) to about 1.6E2 Ohms (water). Similar impedance values are shown in FIG. 7 for skin with and without aqueous solutions. Dry skin exhibited an impedance of about 1.0E6 Ohms at 10 Hz, an electrode gel exhibited an impedance of about 7E4 Ohms at 10 Hz, and a topical lotion exhibited an impedance of about 4E3 Ohms at 10 Hz. The impedance levels for each trended asymptotically to approximately 1.0E3 Ohms at 1 MHz. The electrode gel of FIG. 7 was further evaluated for resistance only, which was generally constant over the range of driving frequencies as shown in FIG. 8. Dry skin exhibited an impedance that transitioned linearly on a log-log plot from about 1.0E8 Ohms at 10 Hz to about 1.0E4 Ohms at 1 MHz as shown in FIG. 9. Finally, FIG. 10 illustrates the resistance from about 10 Hz to about 1 MHz for an electrode gel on a milled wood plank, indicating that surface impedance sensor measurements can discriminate an electrode gel on a foreign material from an electrode gel on skin.

Referring now to FIG. 11, an ultrasound delivery device including the surface impedance sensor 20 of the present invention is illustrated and generally designated 60. In the present embodiment, the ultrasound delivery device 60 is adapted to propagate targeted ultrasonic energy to a sub-dermal region of the skin 40 for cosmetic and/or therapeutic purposes. In other embodiments, however, the ultrasound delivery device 60 can be adapted for use as a medical diagnostic aid, including for example diagnostic sonography. Referring again to FIG. 11, the ultrasound delivery device 60 includes an impedance sensor 20, a transducer 62, a pump 64 and a controller 65 contained within a rigid outer housing 66 to form a self-contained handheld unit. The ultrasound delivery device 60 additionally includes a manually operated control switch 67 that is responsive to the output of the impedance sensor 20 as noted below. The rigid outer housing 66 includes a receptacle for receipt of a gel cartridge 68 in fluid communication with the internal pump 64. The gel cartridge 68 can be one of a plurality of gel cartridges coupled to the ultrasound delivery device 60. In addition, the gel cartridge 68 can include a biocompatible hydrogel, including Signa Gel by Parker Laboratories, Inc., of Fairfield, N.J.

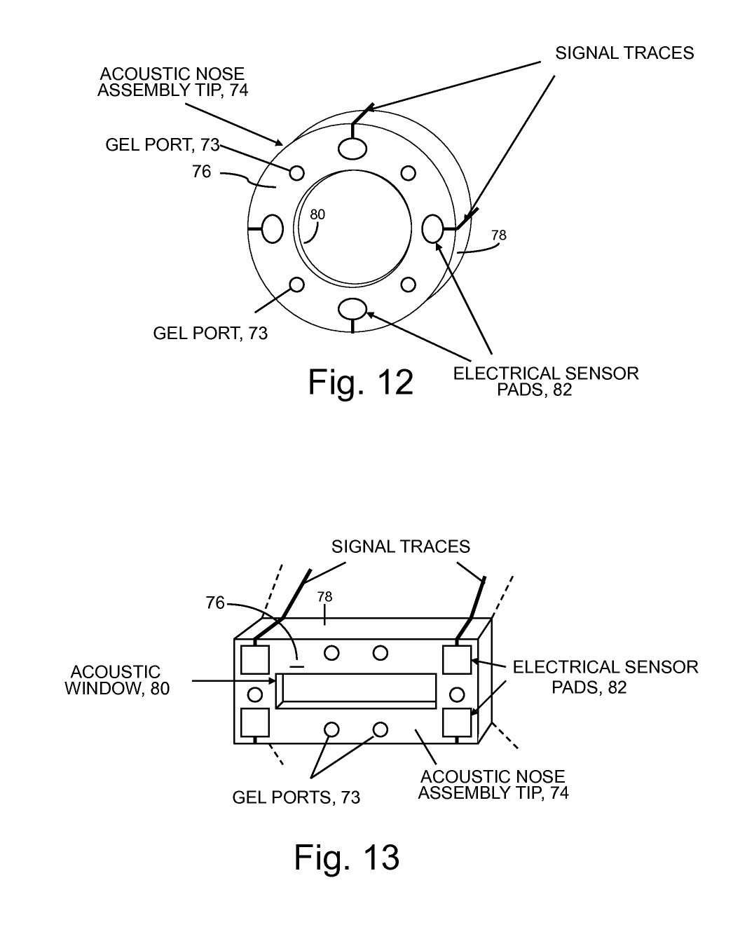

The ultrasound delivery device 60 additionally includes an acoustic nose assembly 71 proximate the transducer 62. The acoustic nose assembly 71 generally includes a wave guide 70, a gel guide 72, and an acoustic nose assembly tip 74. The wave guide 70 can be shaped to focus ultrasonic energy to within the lower epidermal layer. For example, the wave guide 70 can focus ultrasonic energy to within the lower epidermal layer in a line, a spheroid, a spot or any other suitable geometry. The gel guide 72 is concentric with the wave guide 70, being spaced apart from the wave guide 70 for the passage of the transduction gel therebetween. As shown in FIGS. 12-13, the acoustic nose assembly tip 74 can include a skin contacting surface 76 and an upward extending sidewall 78. The skin contacting surface 76 includes an acoustic window 80 to allow the passage of ultrasonic energy therethrough, the acoustic window 80 being optionally circular as shown in FIG. 12 and optionally rectangular as shown in FIG. 13. The skin contacting surface 76 additionally includes one or more gel dispensing ports 73 positioned laterally outward of the acoustic window 80. The gel dispensing ports 73 are circular in the illustrated embodiments, but can be rectangular, curved, arcuate, elongate or any other shape as desired. In addition, the gel dispensing ports 73 can be interposed between adjacent electrical sensor pads 82 as also optionally shown in FIGS. 12-13. The electrical sensor pads 82 can be supported on the skin contacting surface 76 in a fixed spatial relationship. For example, four electrodes 82 are depicted in FIG. 12 as being equidistant from each other at cardinal points laterally outward of the acoustic opening 80. These electrodes 82 include elliptical conducting pads that are electrically isolated from each other and that are electrically coupled to the impedance sensor 20. Also by example, four square electrodes 82 are depicted in FIG. 13. The electrodes 82 are electrically isolated from each other and form a closed circuit when abutting a conductive surface, for example a gel-covered upper epidermal layer as shown in FIG. 11. The acoustic nose assembly tip 74 and the electrodes 82 are translucent to ultrasound waves in the present embodiments to allow the propagation of ultrasonic energy to within the lower epidermal layer. In addition, acoustic nose assembly tip 74 can be formed of a pliable material adapted to conform to the contours of the skin.

In operation, the impedance sensor 20 detects contact with the skin and/or a transduction gel and provides an output substantially as set forth above in connection with FIGS. 1-5. Using the output of the impedance sensor 20, the ultrasound delivery device 60 can administer transduction gel through the gel dispenser ports 73, can propagate ultrasonic energy toward the skin through the acoustic window 80, or both. For example, after activation of the manual switch 67, and where only skin is detected, the ultrasound delivery device 60 can administer transduction gel to the upper epidermal layer. Where both skin and transduction gel is detected, the ultrasound delivery device 60 can activate the transducer 62 to propagate ultrasonic energy to the lower epidermal layer. Where neither skin nor transduction gel is detected, or where a foreign object is detected, the ultrasound delivery device 60 can terminate power to the transducer 62 and the pump 64, or in some instances run the pump 64 in reverse before terminating power. In addition, the impedance sensor 20 can continuously evaluate the impedance across the electrodes 82 as the ultrasound delivery device 60 moves across the skin. For example, the impedance sensor 20 can generate successive impedance profiles as the acoustic nose assembly tip 74 moves along the skin to allow the ultrasound delivery device 60 to incrementally discharge additional gel where needed. In this respect, control of the gel pump 64 includes a negative feedback loop where actual value is the measured impedance profile across the electrodes 82 and the reference value is the reference impedance profile for transduction gel on skin.

Though described above as an ultrasound delivery device, the host device 60 can alternatively include a wide range of other devices. In particular, the host device 60 can include any device where it is desirable to rapidly verify contact with a particular surface, optionally a skin surface. For example, the host device 60 can include a vehicle door handle or a touch sensor, where the output of the surface impedance sensor 20 includes an “enable” command to indicate contact with a human finger. Other host devices are also possible, including for example two-hand trips commonly found in industrial machines and power machinery. As one of skill in the art will appreciate, the use of a surface impedance sensor with a two-hand trip can permit machine activation only after placement of both hands on the trip sensors, as opposed to placement of an errant object against one or both of the trip sensors.

### II. Pulsed Characteristic Classification

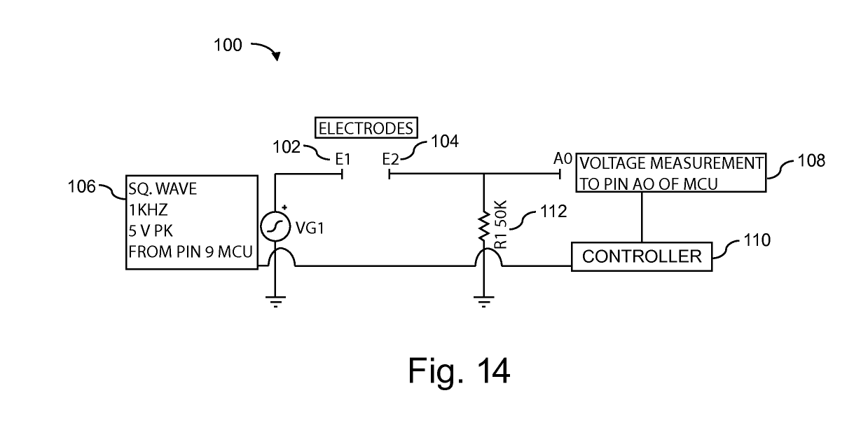

A skin contact sensor in accordance with another embodiment of the invention is illustrated in FIG. 14 and generally designated 100. The skin contact sensor 100 is similar in function to the surface impedance sensor 20 discussed in Part I above, in that the skin contact sensor 100 can be used in conjunction with a host device 60 to rapidly verify contact with a particular surface 40. The skin contact sensor 100 differs from the surface impedance sensor 60 in certain other respects, however. In particular, the skin contact sensor 100 verifies contact with a particular surface 40 based on a measured characteristic(s) of a pulsed voltage, rather than the comparison of a measured impedance profile with a reference impedance profile stored in memory.

Referring now to FIG. 14, the skin contact sensor 100 generally includes first and second electrodes 102, 104, a driver circuit 106 coupled to at least one of the first and second electrodes 102, 104, a measurement circuit 108 coupled to at least the other of the first and second electrodes 102, 104, a controller 110 electrically coupled to the measurement circuit 108 and optionally coupled to the driver circuit 106, and a resistor 112 coupled between the second electrode 104 and ground. As set forth more fully below, the driver circuit 106 is adapted to apply a pulsed signal to the first electrode 102 to generate a pulsed voltage at the second electrodes 104, and the measurement circuit 108 is adapted to measure a characteristic of this pulsed voltage. Based on the measured characteristic, or combination of characteristics, the controller 110 can determine the identity of the surface in contact with the electrodes 102, 104.

The electrodes 102, 104 are similar in structure and function to the electrodes 22, 24 discussed in Part I above. In particular, the electrodes 102, 104 are electrically isolated from each other, optionally being separated by a fixed distance. In one embodiment, the electrodes are 11 mm in length, 2.5 mm in width, and separated by 15.5 mm. The electrode dimensions can vary in other embodiments as desired. The electrodes form a closed circuit when abutting a conductive surface, for example dry skin tissue and gel-covered skin tissue. When used in conjunction with the ultrasound deliver device 60 of FIG. 11, the electrodes 102, 104 can be positioned laterally outward of an acoustic opening as generally depicted in FIGS. 12-13. Further optionally, more than two electrodes 102, 104 can be utilized in some embodiments to potentially increase the versatility of the skin contact sensor 100 and the host ultrasound delivery device 60.

As noted above, the driver circuit 106 is coupled to at least one of the first and second electrodes 102, 104, shown as the first electrode 102 in FIG. 14. In addition, the driver circuit 106 is adapted to drive the at least one electrode with a pulsed signal. The pulsed signal, in turn, generates a pulsed voltage across the first and second electrodes 102, 104. This pulsed voltage will generally vary according to the electrical properties of the surface extending between the first and second electrodes. That is, for a given pulsed signal, the measured pulsed voltage will generally differ among (a) gel-covered skin, (b) dry skin, (c) ultrasound gel but no skin, and (d) air (in which instance the second electrode receives substantially no current).

In the present embodiment, the pulsed signal includes a repeating square wave. In other embodiments, the pulsed signal includes a different waveform. For example, the pulsed signal can include a sawtooth waveform or a sinusoidal waveform. The pulsed signal additionally includes a range of parameters selected by the driver circuit 106, and optionally under the control of the controller 110. The parameters can include, for example, driving frequency, pulse width, and peak amplitude. The driving frequency can be between about 0.01 kHz and about 0.1 MHz inclusive, optionally between about 0.1 kHz and about 10 kHz inclusive, and still further optionally about 1 kHz. The pulse width can be between about 50 microseconds and about 5 milliseconds, optionally about 0.5 milliseconds. The peak amplitude can be between about 0.1 V and about 10 V, optionally between about 1.0 V and 8 V, and further optionally about 5 V. These parameters can vary within or outside of the above ranges, however. These parameters, or other parameters, if desired, are generally kept constant during the evaluation of the surface portion 40.

The measurement circuit 108 is generally adapted to measure one or more characteristics of the pulsed voltage, i.e., the voltage detected at the second electrode 104. A first characteristic includes the difference between the first non-zero value and the last non-zero value for a given pulsed voltage, termed “slope” herein:

slope=leading edge value−trailing edge value (1)

A second characteristic includes the sum of non-zero values for a given pulse, essentially an integral of a portion of the pulsed voltage, termed “area” herein:

area=Σnon-zero values (2)

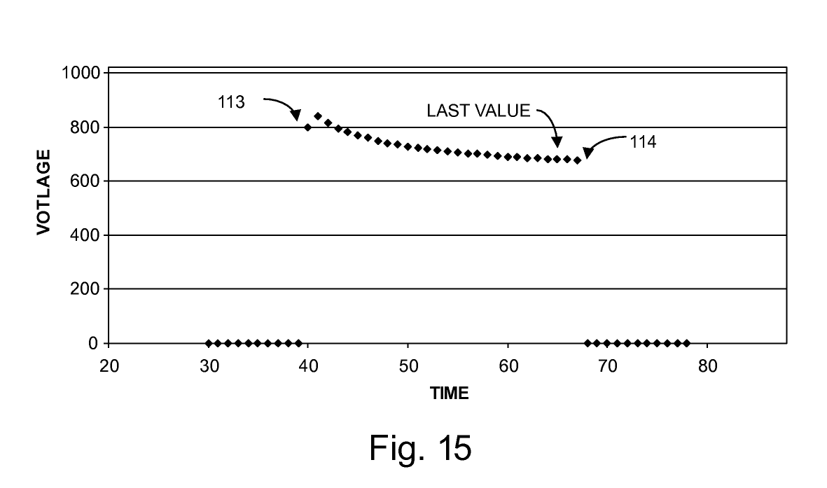

In the present embodiment, the sum includes the first non-zero value and twenty-four subsequent values. In this embodiment, the twenty-fifth value is the “last value”. To further illustrate, an exemplary pulsed voltage for gel-covered skin is illustrated in FIG. 15. The pulsed voltage includes a leading portion 113 and a trailing portion 114. Each unit of time on the x-axis corresponds to 0.01 milliseconds, and each unit of voltage on the y-axis corresponds to 5 mV. Using equation (1) above, the slope for the pulsed voltage in FIG. 15 is approximately 122 units, corresponding to 0.61 V. Using equation (2) above, the area for the pulsed voltage in FIG. 15 is approximately 20,308 units, corresponding to 101.5 V.

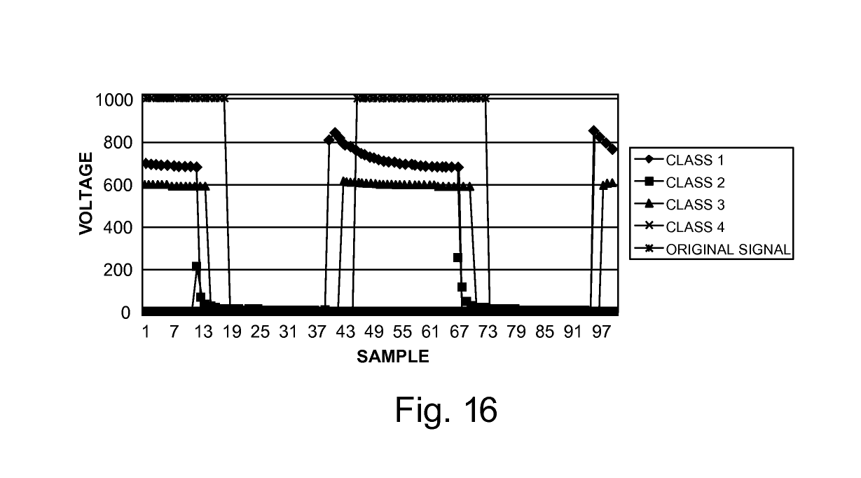

In addition to the pulsed voltage depicted in FIG. 15, the measurement circuit 108 is adapted to determine the area and the slope for other pulsed voltages, including the pulsed voltages depicted in FIG. 16. The driving signal in FIG. 16 includes a repeating 1 kHz square wave having a 5V peak amplitude and a 0.5 millisecond pulse width (“Original Signal”). The pulsed voltages correspond to a) gel-covered skin (“Class 1”); b) dry skin (“Class 2”); c) gel and no skin (“Class 3”); and d) neither skin nor gel (“Class 4”). Because each surface includes unique electrical properties, the surfaces under evaluation can be distinguished from one another based on the first characteristic, the second characteristic, a combination of the first and second characteristics, or other characteristics not discussed above. The controller 110 is generally adapted to determine, using the characteristic(s), the identity of the surface portion 40, optionally with reference to a classification table stored in computer readable memory. For example, the following classification table includes a listing of surface portions according to slope and area:

| Class | Surface Portion | Slope | Area |
|---|---|---|---|
| 1 | gel and skin | >60 | >17,000 |
| 2 | skin (no gel) | >60 | <17,000 |
| 3 | gel (no skin) | <60 | >10,000 |
| 4 | no gel and no skin | <60 | <10,000 |

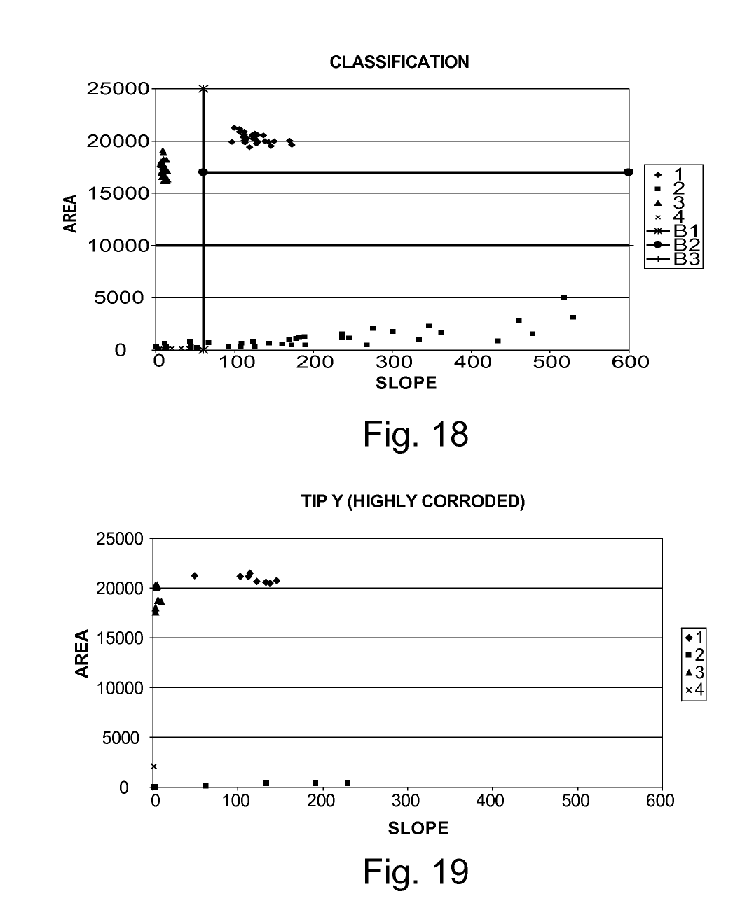

A classification graph illustrating the above four classifications is illustrated in FIG. 18. The slope threshold is depicted as “B1”, corresponding to about 6% of the amplitude of the pulsed signal. Two area thresholds are indicated. The upper area threshold is depicted as “B2”, corresponding to about seventeen times the peak amplitude of the pulsed signal. The lower area threshold is depicted as “B3”, corresponding to about ten times the peak amplitude of the pulsed signal.

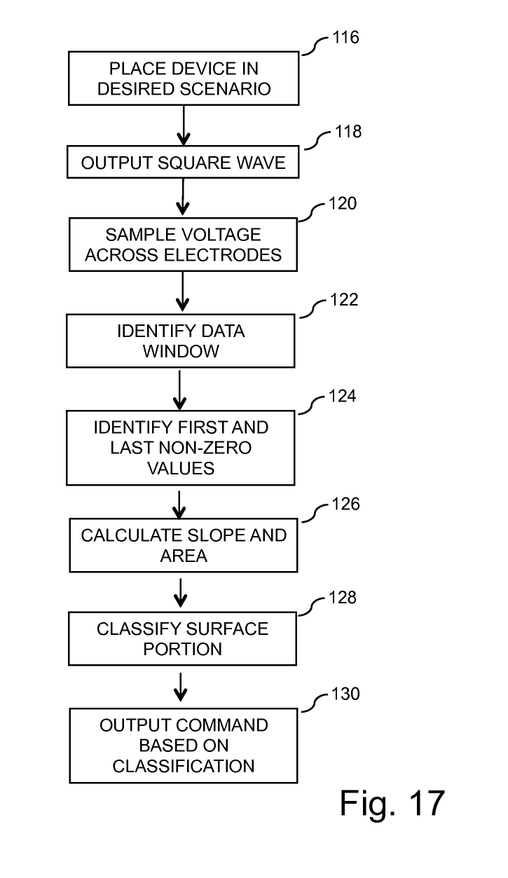

Further with respect to the present embodiment, a method for identifying a surface portion is illustrated in the flow chart of FIG. 17. At step 116, the skin contact sensor electrodes 102, 104 are placed in contact with a surface portion 40. At step 118, the driver circuit outputs a square wave at a single frequency and amplitude. In the above embodiment, the square wave includes an amplitude of 5V and a frequency of 1 kHz. At step 120, voltage across the electrodes 102, 104 is sampled at a desired sampling frequency. In the above embodiment, the sampling frequency is 56 kHz. At step 122, a usable data window is identified, and at step 124, the leading edge and trailing edge (i.e., first and last non-zero) within the usable data window is determined. At step 126, a slope and an area are determined using equations (1) and (2) above. At step 128, and using a classification table stored in computer readable memory, the identity of the surface portion is determined. Lastly, at step 130, the skin contact sensor 100 or a host device controller outputs a command based on the identity of the surface portion. For example, where skin is identified, additional ultrasound gel can be dispensed. Where skin and gel is identified, the transducer 62 can be activated. In the absence of skin, no gel can be dispensed, and the transducer 62 can remain off.

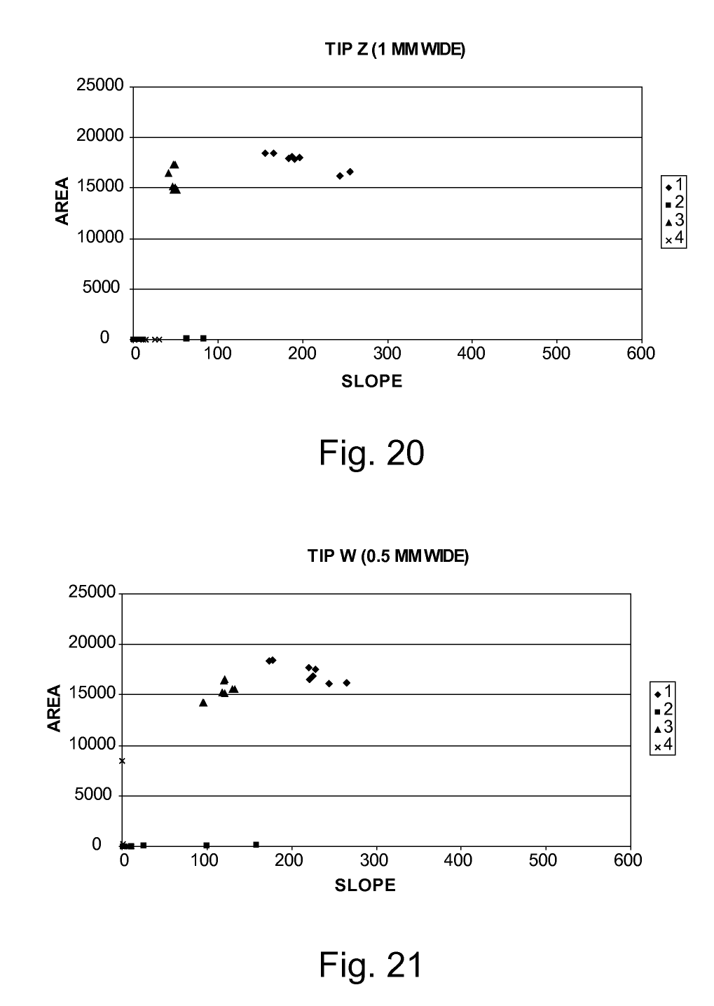

To reiterate, the present embodiment provides a skin contact sensor 100 for use in conjunction with a classification table stored in memory to rapidly identify a surface portion in contact with two or more electrodes, optionally in less than 6 milliseconds in some embodiments, and with a demonstrated accuracy of greater than 94%. The present embodiment also has versatility with corroded electrodes. In one example, non-corroded electrodes were provided, including a length of 11 mm, a width of 2.5 mm, and a gam of 15.5 mm. The electrodes were corroded by submerging in water with high total dissolved solids (TDS) and by applying a DC signal of 32 volts and 0.06 amps for ten minutes. Thirty-two measurements were taken over the four classifications noted above. The skin contact sensor 100 demonstrated an accuracy of almost 97% in this trial, with the results depicted in FIG. 19. The accuracy of the skin contact sensor 100 diminished somewhat with electrodes having a width of less than 1 mm. In particular, thirty-two measurements for electrodes having a 1 mm width (reduced from 2.5 mm) demonstrated an accuracy of about 94%, while thirty-two measurements for electrodes having a 0.5 mm width demonstrated an accuracy of about 63%. The results of these measurements are depicted in FIGS. 20 and 21. The most noticeable outcome of changing the width was the proximity of the class 3 data (gel only) to class 1 data (gel-covered skin), making these surfaces more difficult to distinguish.

Accordingly, the skin contact sensor and method of the present embodiment provide for the rapid identification of a surface portion with improved accuracy and with minimal hardware and computing resources. The skin contact sensor and method include a resistance to corrosion, with some flexibility in the shape and the size of the electrodes. The skin contact sensor and method can also meet the requirements of IEC 60601 for medical electrical equipment by providing a current less than 100 μA. The skin contact sensor and method can also be implemented in devices unrelated to medical applications, including vehicle door handles and two-hand trips.

The above description is that of current embodiments of the invention. Various alterations and changes can be made without departing from the spirit and broader aspects of the invention as defined in the appended claims, which are to be interpreted in accordance with the principles of patent law including the doctrine of equivalents. This disclosure is presented for illustrative purposes and should not be interpreted as an exhaustive description of all embodiments of the invention or to limit the scope of the claims to the specific elements illustrated or described in connection with these embodiments. Any reference to elements in the singular, for example, using the articles “a,” “an,” “the,” or “said,” is not to be construed as limiting the element to the singular.

1. A method comprising:
applying first and second spaced apart electrodes to a surface portion;
driving the first and second electrodes at a plurality of frequencies;
measuring the surface impedance across the electrodes for each of the plurality of driving frequencies to generate a measured surface impedance profile; and
correlating the measured surface impedance profile with one of a plurality of reference surface impedance profiles to identify the surface portion.

2. The method according to claim 1 wherein identifying the surface portion includes distinguishing among a plurality of surfaces.

3. The method according to claim 1 wherein measuring the surface impedance includes measuring the complex surface impedance.

4. The method according to claim 1 wherein the plurality of driving frequencies includes about 10 Hz and about 1 MHz.

5. The method according to claim 1 wherein each of the plurality of impedance profiles correspond to a unique surface.

6. The method according to claim 1 wherein the surface portion is non-dimensionally stable.

7. The method according to claim 1 wherein the surface portion includes human tissue.

8. The method according to claim 1 wherein correlating a measured surface impedance profile is performed with a controller.

9. The method according to claim 8 wherein the controller is housed within an ultrasound gel dispenser.

10. The method according to claim 8 wherein the ultrasound gel dispenser is responsive to the output of the controller.

11. The method according to claim 8 wherein the ultrasound gel dispenser is housed within a therapeutic ultrasound device.

12. A surface impedance sensor comprising:
first and second electrodes;
a driver circuit adapted to drive the first and second electrodes at a plurality of driving frequencies;
a detection circuit to measure the impedance across the first and second spaced apart electrodes for each of the plurality of driving frequencies; and
a controller electrically coupled to the detection circuit and adapted to compare the detected impedance against a plurality of impedance profiles.

13. The surface impedance sensor of claim 12, wherein the detected impedance is used to indicate placement of the electrodes against a surface.

14. The surface impedance sensor of claim 12, wherein the detected impedance is used to distinguish among a plurality of surfaces.

15. The surface impedance sensor of claim 12, wherein the detection circuit is adapted to measure complex impedance for each of the plurality of frequencies.

16. The surface impedance sensor of claim 12 wherein measured surface impedance forms an impedance curve, the controller including pattern recognition logic to correlate the impedance curve with one of the plurality of impedance profiles.

17. The surface impedance sensor of claim 12 wherein the controller is adapted to provide an output indicative of the presence or absence of a surface in contact with the first and second electrodes.

18. The surface impedance sensor of claim 12 wherein the controller is adapted to provide an output indicative of the identity of the surface in contact with the first and second electrodes.

19. The surface impedance sensor of claim 18 wherein the controller is adapted to provide the output to an ultrasound delivery device.

20. The surface impedance sensor of claim 19, wherein the electrodes are translucent to ultrasound waves.

21. The surface impedance sensor of claim 12 wherein the driver circuit is adapted to drive the first and second electrodes across a first frequency between about 1 Hz and about 100 Hz and a second frequency between about 0.1 MHz and about 10 MHz.

22. A skin contact sensor comprising:
first and second electrodes;
a driver circuit adapted to generate a pulsed voltage across the first and second electrodes;
a measurement circuit coupled to at least one of the first and second electrodes and adapted to measure a characteristic of the pulsed voltage; and
a controller electrically coupled to the measurement circuit and adapted to determine the identity of a surface portion in contact with the first and second electrodes based on the measured characteristic.

23. The skin contact sensor of claim 22 wherein the driver circuit is adapted to apply a pulsed signal to the first electrode.

24. The skin contact sensor of claim 23 wherein the pulsed signal includes a repeating square wave.

25. The skin contact sensor of claim 23 wherein the pulsed signal includes a frequency of between about 0.1 kHz and about 10 kHz, inclusive.

26. The skin contact sensor of claim 23 wherein the pulsed signal includes a frequency of about 1 kHz.

27. The skin contact sensor of claim 23 wherein the pulsed signal includes a pulse width of between approximately 50 microseconds and 5 milliseconds, inclusive.

28. The skin contact sensor of claim 23 wherein the pulsed signal includes a pulse width of approximately 0.5 milliseconds.

29. The skin contact sensor of claim 23 wherein the measurement circuit is adapted to sample the pulsed voltage at a rate of at least 50 kHz.

30. The skin contact sensor of claim 22 wherein the characteristic includes the difference between first and last non-zero portions of the pulsed voltage.

31. The skin contact sensor of claim 22 wherein the characteristic includes the summation of a plurality of non-zero portions of the pulsed voltage.

32. The skin contact sensor of claim 22 wherein the controller is adapted to provide an output based on the identity of the surface portion.

33. A method comprising:
applying first and second electrodes to a surface portion;
driving the first electrode with a pulsed signal;
measuring a voltage across the second electrode;
determining first and second characteristics of the measured voltage; and
using the determined characteristics, identifying the surface portion.

34. The method according to claim 33 wherein the pulsed signal includes a repeating square wave.

35. The method according to claim 33 wherein the pulsed signal includes a frequency of between about 0.1 kHz and about 10 kHz, inclusive.

36. The method according to claim 33 wherein the pulsed signal includes a peak amplitude of between about 0.5 V and about 10 V, inclusive.

37. The method according to claim 33 wherein the measured voltage is sampled at a rate of at least 50 kHz.

38. The method according to claim 33 wherein the first characteristic includes the difference between two non-zero portions of the measured voltage.

39. The method according to claim 33 wherein the second characteristic includes a summation of at least two non-zero portions of the measured voltage.

40. The method according to claim 33 wherein the measured voltage includes a measured pulse, and wherein the surface portion is identified based on:
the difference between first and last non-zero portions of the measured pulse being greater than about 6% of the amplitude of the pulsed signal; and
the summation of a plurality of non-zero portions of the measured pulse being at least seventeen times the amplitude of the pulsed signal.

41. The method according to claim 33 wherein the pulsed signal includes a pulse width of between approximately 50 microseconds and 5 milliseconds, inclusive.

42. The method according to claim 33 wherein the pulsed signal includes a pulse width of approximately 0.5 milliseconds.

43. The method according to claim 33 wherein the pulsed signal includes a current of less than 100 μA.
